(19) United States
(12) Patent Application Publication
(10) Pub. No.: US 2022/0133285 A1
(43) Pub. Date: May 5, 2022

(54) BIOPSY DEVICE FOR USE WITH ENDOSCOPE

(71) Applicant: BOSTON SCIENTIFIC SCIMED, INC., Maple Grove, MN (US)

(72) Inventors: Michael S. H. Chu, Brookline, MA (US); Sacha Tang, Lowell, MA (US); Pat S. Phongsavanh, Blackstone, MA (US)

(73) Assignee: BOSTON SCIENTIFIC SCIMED, INC., Maple Grove, MN (US)

(21) Appl. No.: 17/518,975

(22) Filed: Nov. 4, 2021

Related U.S. Application Data
(60) Provisional application No. 63/110,139, filed on Nov. 5, 2020.

(51) Int. Cl. A61B 10/04; A61B 10/02; A61B 10/06

(52) U.S. Cl. CPC A61B 10/04; A61B 2010/0208; A61B 10/06; A61B 10/0266

(57) ABSTRACT

A biopsy device adapted for use with an endoscope includes an adaptor that is adapted to be secured relative to an endoscope. The biopsy device includes a pair of jaws that are pivotably disposed relative to the adaptor. An actuator is operably coupled with the first jaw and the second jaw and extends proximally therefrom. The actuator is adapted to move the first jaw and the second jaw relative to the biopsy device housing.

### CROSS REFERENCE TO RELATED APPLICATIONS

This application claims the benefit of and priority to U.S. Provisional Patent Application Ser. No. 63/110,139 filed on Nov. 5, 2020, the disclosure of which is incorporated herein by reference.

### TECHNICAL FIELD

The present disclosure pertains to medical devices for removing tissue. More particularly, the present disclosure pertains to medical devices usable with an endoscope for removing tissue.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. As an example, a biopsy device adapted for use with an endoscope includes an adaptor that is adapted to be secured relative to the endoscope. The adaptor includes an inner surface and an outer surface. A pair of jaws is pivotably disposed relative to the adaptor. An actuator is operably coupled with the pair of jaws and extends proximally therefrom. The actuator is adapted to move the pair of jaws relative to the adaptor.

Alternatively or additionally, the adaptor may be adapted to be moved in a first axial direction to open the pair of jaws and to be moved in an opposing second axial direction to close the jaws.

Alternatively or additionally, the pair of jaws may be biased to a first position, and the actuator is adapted to move the pair of jaws against a biasing force.

Alternatively or additionally, the adaptor may include a first pivot point along a first side of the biopsy device housing and a second pivot point along a second side of the biopsy device housing.

Alternatively or additionally, the first pivot point and the second pivot point may be disposed on the inner surface of the adaptor.

Alternatively or additionally, the first pivot point and the second pivot may be disposed on the outer surface of the adaptor.

Alternatively or additionally, the actuator may include a first actuator member secured relative to the first jaw and a second actuator member secured relative to the second jaw such that applying an axial force to the first actuator member and/or the second actuator member causes the first jaw and/or the second jaw to pivot relative to the biopsy device housing.

Alternatively or additionally, the first jaw may include a first actuator arm and the second jaw may include a second actuator arm, with the first actuator member secured to the first actuator arm and the second actuation member secured to the second actuator arm.

Alternatively or additionally, the adaptor may include a first alignment feature that accommodates the first actuator member extending therethrough and a second alignment feature that accommodates the second actuator member extending therethrough.

Alternatively or additionally, the biopsy device may further include a coupler adapted to releasably secure the biopsy device housing relative to the endoscope.

Alternatively or additionally, the biopsy device may further include an actuator handle operably coupled with the actuator member.

Alternatively or additionally, the actuator handle may include a first member slidingly secured relative to a second member, with the actuator securable to the first member, wherein moving the first member relative to the second member may cause the actuator to open or close the pair of jaws.

Alternatively or additionally, the actuator handle may include a rotatable wheel, with the actuator securable to the rotatable wheel, and rotating the wheel may cause the actuator to open or close the pair of jaws.

As another example, a biopsy device is adapted for use with an endoscope. The biopsy device includes an adaptor adapted to be secured relative to the endoscope. A first jaw is pivotably secured to the adaptor. A second jaw is pivotably secured to the adaptor, and the first jaw and the second jaw together are pivotably to a closed configuration in which the first jaw and the second jaw cooperate to cut tissue. A first actuator member is secured to the first jaw such that moving the first actuator member causes the first jaw to pivot relative to the adaptor and a second actuator member is secured to the second jaw such that moving the second actuator member causes the second jaw to pivot relative to the adaptor. An actuator handle is operably coupled with the first actuator member and the second actuator member such that manipulating the actuator handle causes the first jaw and the second jaw to pivot relative to the adaptor.

Alternatively or additionally, the first actuator member and the second actuator member may extend proximally to the actuator handle.

Alternatively or additionally, the first actuator member and the second actuator member may be coupled together, and only one of the first actuator member and the second member may extend proximally to the actuator handle.

Alternatively or additionally, the actuator handle may include a first handle slidingly secured relative to a second handle, with at least one of the first actuator member and the second actuator handle securable to the first member, wherein moving the first handle relative to the second handle may cause the actuator to open or close the pair of jaws.

Alternatively or additionally, the actuator handle may include a rotatable wheel, with the first actuator member and the second actuator member securable to the rotatable wheel, and rotating the wheel may cause the actuator to open or close the pair of jaws.

As another example, a medical device is adapted for use with an access device. The medical device includes an adaptor adapted to be secured relative to an access device. A pair of jaws is pivotably secured to the adaptor and are movable between an open position in which each of the pair of jaws pivots away from each other and a cutting position in which each of the pair of jaws pivots toward each other. A spring biases the pair of jaws towards one of the open position and the cutting position. An actuator is operably coupled with the pair of jaws and extends proximally therefrom and is adapted to move the pair of jaws against a biasing force applied by the spring.

Alternatively or additionally, the medical device may further include an actuator handle operably coupled with the actuator.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a perspective view of an illustrative endoscope;

FIG. 2 is a perspective view of an illustrative biopsy device usable with the illustrative endoscope of FIG. 1;

FIG. 3 is a perspective view of a portion of the illustrative biopsy device of FIG. 2, shown with the jaws in a closed configuration;

FIG. 4 is a cross-sectional view taken along line 4-4 of FIG. 3;

FIG. 5 is a schematic cross-sectional view of the illustrative biopsy device;

FIG. 6 is a perspective view of a pair of jaws forming part of the illustrative biopsy device of FIG. 2, with the pair of jaws shown in a closed configuration;

FIG. 7 is a perspective view of a pair of jaws forming part of the illustrative biopsy device of FIG. 2, with the pair of jaws shown in an open configuration;

FIG. 8 is a perspective view of a jaw forming part of the illustrative biopsy device of FIG. 2;

FIG. 9 is a perspective view of a portion of the illustrative biopsy device of FIG. 2, shown with the jaws in an open configuration;

FIG. 10 is a side view of an illustrative biopsy device;

FIG. 11 is a top view of the illustrative biopsy device of FIG. 10;

FIG. 12 is an enlarged view of a portion of the illustrative biopsy device of FIG. 10;

FIG. 13 is a side view of an illustrative biopsy device, shown with the jaws in a closed configuration;

FIG. 14 is an end view of the illustrative biopsy device of FIG. 13;

FIG. 15 is a side view of the illustrative biopsy device of FIG. 13, shown with the jaws in an open configuration;

FIG. 16 is an end view of the illustrative biopsy device of FIG. 15;

FIGS. 17 through 19 are side views of illustrative extension hook wires;

FIGS. 20 and 21 are front and side views, respectively, of an illustrative pair of jaws;

FIGS. 22 through 24 are perspective, top and end views, respectively, of an illustrative adaptor and coupler;

FIGS. 25 through 27 are perspective, bottom and end views, respectively, of an illustrative biopsy device;

FIGS. 28 and 29 are schematic views of illustrative torsion springs;

FIG. 30 is a side view of an illustrative pair of jaws having a normally open position;

FIGS. 31 and 32 are schematic views of cutting patterns that may be used to form the illustrative pair of jaws of FIG. 30;

FIGS. 33 and 34 are side and end views, respectively, of an illustrative biopsy device including the illustrative pair of jaws of FIG. 30;

FIG. 35 is a perspective view of an illustrative adaptor and coupler usable in the illustrative biopsy device of FIGS. 33 and 34;

FIG. 36 is a perspective view of an illustrative biopsy device; and

FIG. 37 is a perspective view of an illustrative biopsy device.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

The disclosure pertains to manipulating, cutting, grabbing, ligating, and/or otherwise treating tissue. In some instances, the devices, systems and/or methods discussed herein may be utilized during endoscopic mucosal resection (EMR) and/or endoscopic submucosal dissection (ESD) procedures. In some cases, EMR and ESD include endoluminal placement of one or more devices for grasping and cutting tissue proximate to a target area within the body of a patient. Placement of the one or more medical devices may be via a catheter, scope (endoscope, bronchoscope, colonoscope, gastroscope, duodenoscope, etc.), tube, or sheath, inserted into the GI tract via a natural orifice or incision. The orifice can be, for example, the nose, mouth, or anus, and the placement can be in any portion of the GI tract, including the esophagus, stomach, duodenum, large intestine, or small intestine. Placement also can be in other organs reachable via the GI tract. The patient's tissue may be grasped using suction from one or more medical devices and/or a grasper, and then the tissue may be cut by a cutting device for subsequent removal from the patient's body.

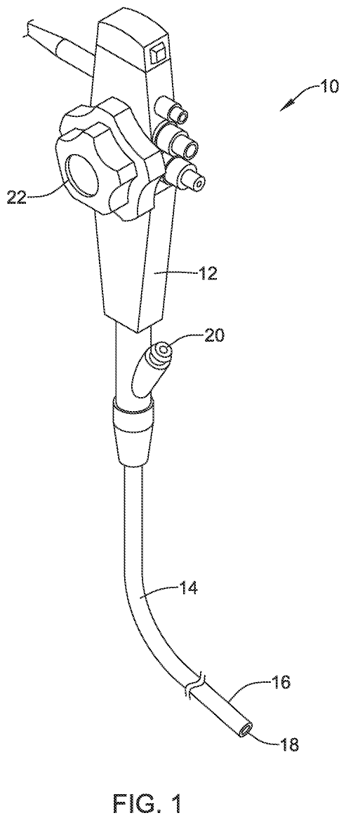

In some cases, an endoscope may be used. An example endoscope 10 is illustrated in FIG. 1. The endoscope 10 may be any of a number of types of endoscopes or related medical devices usually identified by the particular anatomy desired to be reached. For example, the endoscope 10 may be a bronchoscope, colonoscope, duodenoscope, esophagoscope, guidetubes, introducers (without or without vision or visualization capabilities), or any other type of endoscope or related medical device. The endoscope 10 may include a handpiece 12 and an elongate shaft 14 extending distally from the handpiece 12 to a distal tip region 16. The handpiece 12, for example, may include a number of controls such as one or more control knobs 22. The shaft 14 may include a lumen defining a working channel 18 extending through the shaft 14. Although the endoscope 10 is depicted with a single working channel in FIG. 1, it can be appreciated that in other embodiments, the endoscope 10 may include multiple working channels, as desired. A biopsy port 20 may be disposed adjacent to the shaft 14 and the biopsy port 20 may provide access to the working channel 18.

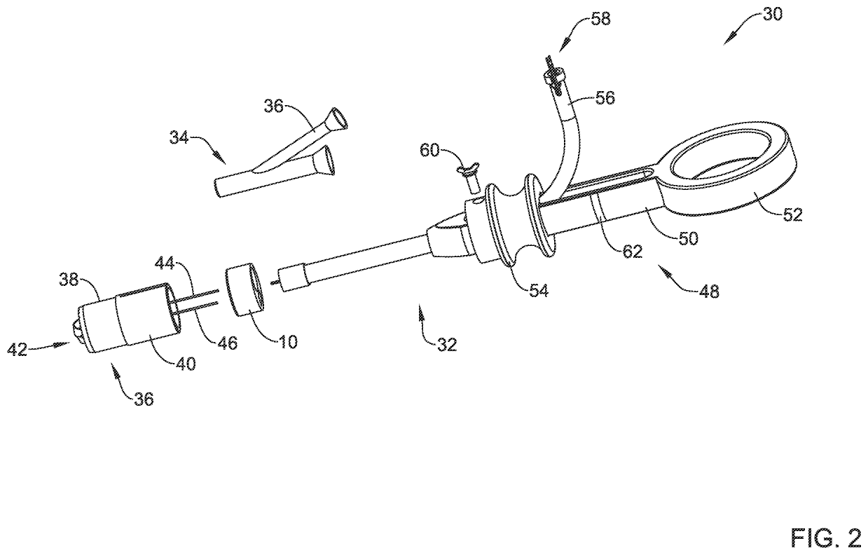

FIG. 2 is a perspective view of an illustrative assembly 30. The illustrative assembly 30 may include a biopsy device 32 used in conjunction with the endoscope 10. It will be appreciated that portions of the endoscope 10 are shown schematically in FIG. 2. For example, in some instances, the endoscope 10 may include a Y connector 34 with a side port 36. While described as an endoscope, it will be appreciated that the endoscope 10 may also be any of a colonoscope, a sigmoidoscope, an enteroscope, a cystoscope, a ureteroscope, a flexible ureteroscope or the like.

The biopsy device 32 includes a distal assembly 36. As will be discussed in greater detail with respect to subsequent Figures, the distal assembly 36 may include an adaptor 38 that is adapted to be secured to the distal tip region 16 of the endoscope 10. A resilient elastomeric coupler 40 is adapted to releasably secure the adaptor 38 to the distal tip region 16 of the endoscope 10. The distal assembly 36 includes a pair of jaws 42 that are adapted for cutting or otherwise incising tissue in order to obtain a biopsy sample, for example. The pair of jaws 42 may be adapted to move between an open position, in which the pair of jaws 42 are not positioned to cut tissue, and a closed position in which the pair of jaws 42 interact together to cut tissue. In some cases, one of the pair of jaws 42 may move over the other of the pair of jaws 42 such that a shear cut is provided. In some cases, the pair of jaws 42 may be configured such that the cutting edge of one of the pair of jaws 42 directly contacts the cutting edge of the other of the pair of jaws 42. The cutting edges of each of the pair of jaws 42 may be serrated, for example. In some cases, one of the pair of jaws 42 has a smooth cutting edge while the other of the pair of jaws 42 has a flat surface against which the smooth cutting edge interacts in order to cut tissue.

The biopsy device 30 includes a first actuator member 44 and a second actuator member 46. The first actuator member 44 may be secured to one of the pair of jaws 42 while the second actuator member 46 may be secured to the other of the pair of jaws 42. Axial movement of the first actuator member 44 and/or the second actuator member 46 may cause relative movement of the pair of jaws 42 between their closed configuration and their open configuration, for example. The first actuator member 44 and the second actuator member 46 each extend proximally from the distal assembly 36 towards an actuator handle 48. The actuator handle 48 may take a variety of different forms, as along as the actuator handle 48 is adapted to enable a user to axially translate the first actuator member 44 and/or the second actuator member 46 relative to the distal assembly 36 and thus cause a corresponding movement of the pair of jaws 42. While the biopsy device 30 as shown in FIG. 2 shows the first actuator member 44 and the second actuator member 46 both extending proximally to the actuator handle 48, this is not necessary in all cases. For example, in some cases, only a single actuator extends proximally to the actuator handle 48.

As illustrated, the actuator handle 48 includes a handle body 50 having a thumb ring 52 as well as a finger ring 54 that may be slidingly disposed relative to the handle body 50. The first actuator member 44 and the second actuator member 46 extend proximally through the finger ring 54 and into and thru an angled tube 56. The first actuator member 44 and the second actuator member 46 are crimped together via a crimp 58. As a result, the first actuator member 44 and the second actuator member 46 move together in unison. A fastener 60 may be threadedly engaged with the finger ring 54 in order to secure the first actuator member 44 and the second actuator member 46 relative to the finger ring 54 such that movement of the finger ring 54 relative to the handle body 50 causes corresponding movement of the pair of jaws 42. In some cases, the handle body 50 may include a proximal marker 62 that reminds the user how far proximally the finger ring 54 should be moved relative to the handle body 50. The proximal marker 62 may be a colored band, or may be a tab or other structure that extends far enough from the handle body 50 so that the user can feel the presence of the proximal marker 62 while moving the finger ring 54 relative to the handle body 50.

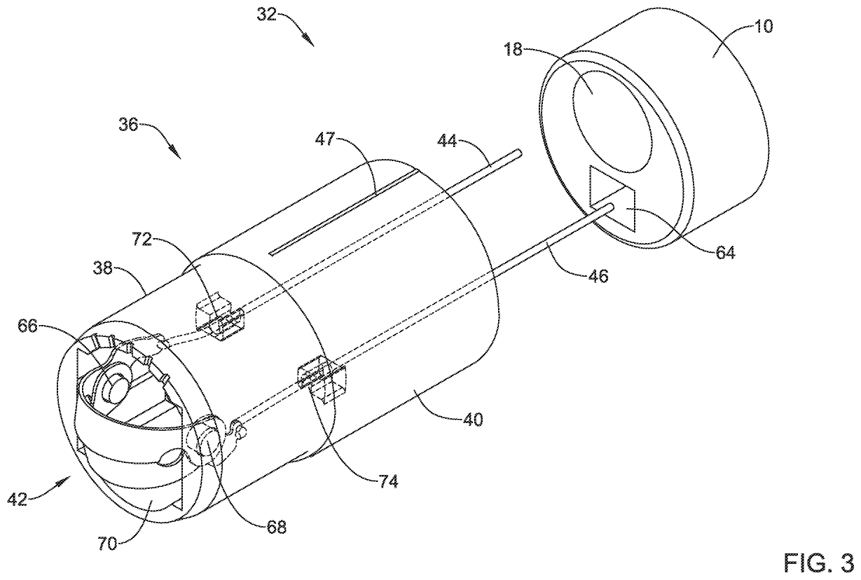

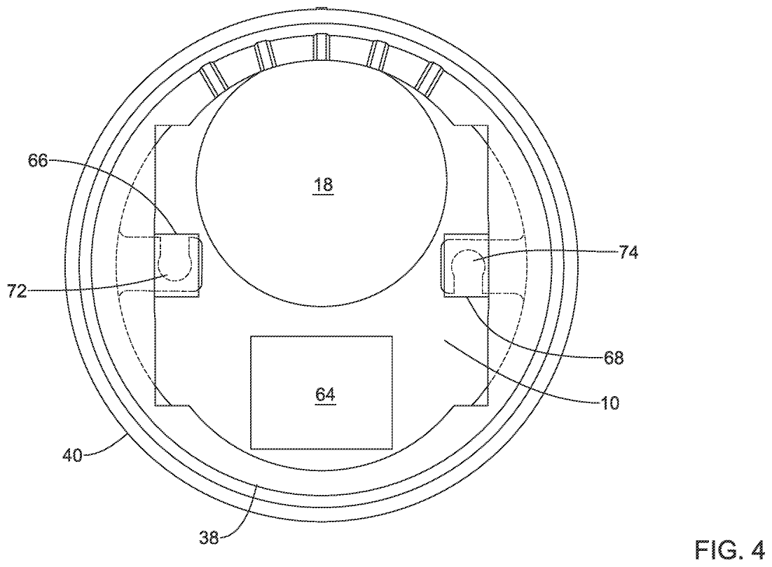

FIG. 3 is a perspective view of a portion of the illustrative biopsy device 32 with the pair of jaws 42 shown in a closed position while FIG. 4 is a cross-sectional view of the illustrative biopsy device 32 secured relative to the endoscope 10, taken along line 4-4 of FIG. 3. This position corresponds to the relative position of each of the pair of jaws 42 relative to each other when the jaws 42 are cutting tissue. In some cases, as shown, the adaptor 38 is made from a translucent or even transparent polymer in order to permit viewing through the adaptor 38. Examples of suitable transparent polymers include polycarbonate, polystyrene, polymethyl methacrylate (PMMA) and acrylonitrile butadiene styrene (ABS) clear. While not shown in FIG. 1, in some cases the endoscope 10 may include a camera 64 that is disposed adjacent the working channel 18. It will be appreciated that the video and other images provided by the camera 64. The coupler 40 may be formed of any suitable polymeric material that exhibits sufficient stretchiness to enable the coupler 40 to be stretched over both the proximal end of the adaptor 38 as well as the distal tip region 16 of the endoscope 10. Examples of suitable polymers include silicone, rubber, polyurethane and thermoplastic elastomers (TPE). The coupler 40 may include an alignment marker 47 that, as will be discussed, may be used to align with the working channel 18 of the endoscope 10.

The adaptor 38 includes hinge pins 66 and 68. The hinge pins 66 and 68 are situated 180 degrees apart, one on each side of the adaptor 38. As shown in FIG. 3, the hinge pins 66 and 68 are disposed on an interior surface 70 of the adaptor 38. In other cases, as will be discussed, the hinge pins 66 and 68 may instead be disposed on an exterior surface of the adaptor 38. In some cases, the hinge pins 66 and 68 are molded hinge pins, formed when the adaptor 38 is formed. In some cases, the hinge pins 66 and 68 may instead each be part of a fastener, such as the shaft of a bolt or screw, a rivet or the like, that fasten the pair of jaws 42 to the adaptor 38 while permitting the pair of jaws 42 to rotate relative to the adaptor 38.

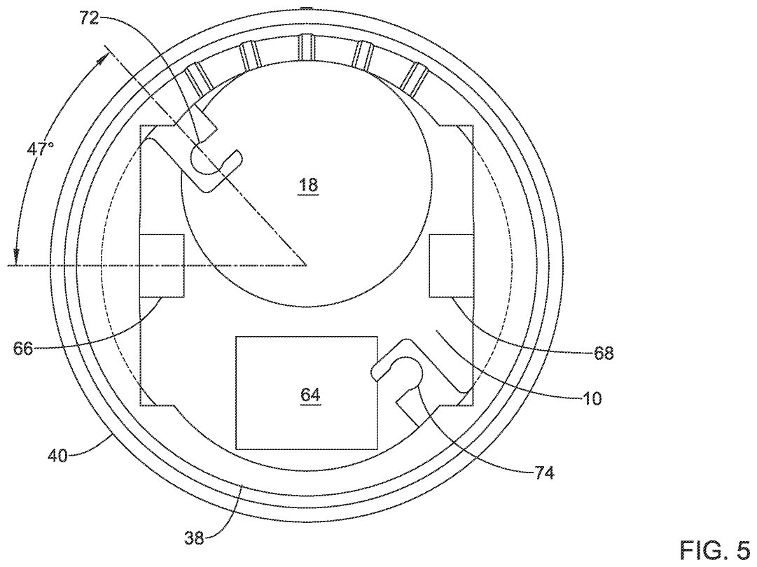

The adaptor 38 includes guide holes 72 and 74 that are each adapted to accommodate and locate the first actuator member 44 and the second actuator member 46, respectively. In some cases, as shown for example in FIG. 4, the guide hole 72 lines up with the first hinge pin 66 and the guide hole 74 lines up with the second hinge pin 68. Additional guide holes (not shown) may be located within the adaptor 38 in order to guide or direct the actuator members 44 and 46 against the inner wall and out of view of the camera 64. In some cases, as shown for example in FIG. 5, the guide holes 72, 74 may be rotated relative to a horizontal plane extending through the hinge pins 66 and 68. In some instances, this may allow the hinge pins 66 and 68 to be moldable, as the guide holes 72 and 74 are not in the way. The additional height H between the guide hole 72 and the hinge pin 66 allows for additional degrees of rotation. In some cases, the height H provides a pull wire angle/moment arm in which a greater force can be applied to the closed jaw for severing tough tissue, for example. It will be appreciated that when the guide holes 72 and 74 are aligned with the hinge pins 66 and 68, any additional force applied by the user will not move the pair of jaws 42.

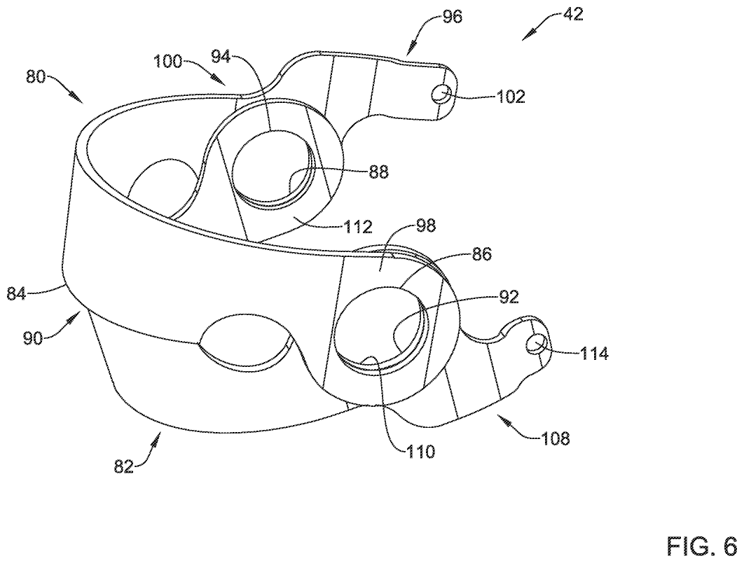

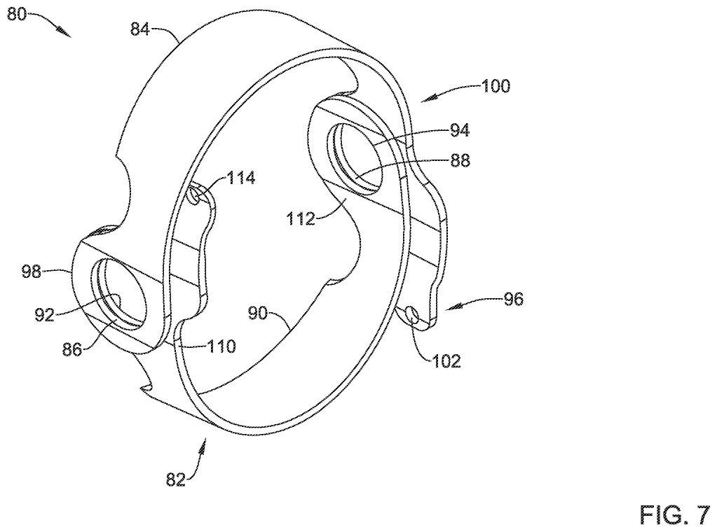

FIGS. 6 and 7 provide additional views of the pair of jaws 42. In FIG. 6, the pair of jaws 42 is shown in a closed configuration while in FIG. 7 the pair of jaws 42 is shown in an open configuration. The pair of jaws 42 includes a first jaw 80 and a second jaw 82. In some cases, the first jaw 80 may be slightly larger than the second jaw 82, which allows the jaws 80, 82 to slide past each other when the jaws move into their closed configuration. In some cases, the first jaw 80 and the second jaw 82 may be made to be the same size. The first jaw 80 includes a cutting edge 84 and a pair of hinge holes 86 and 88. Similarly, the second jaw 82 includes a cutting edge 90 and a pair of hinge holes 92 and 94. The cutting edges 84 and 90 may be sharp blades, or the cutting edges 84 and 90 may be serrated edges. It will be appreciated that the hinge holes 88 and 94 are adapted to fit over the first hinge pin 66 while the hinge holes 86 and 92 are adapted to fit over the second hinge pin 68. In assembling the biopsy device 32, in some cases the first jaw 80 and the second jaw 82 may be compressed slightly from a relaxed profile to allow placement of the first jaw 80 and the second jaw 82 relative to the first hinge pin 66 and the second hinge pin 68. Releasing the first jaw 80 and the second jaw 82 allows the first jaw 80 and the second jaw 82 to relax back to their relaxed profile allows the hinge pins 66 and 68 to secure the first jaw 80 and the second jaw 82 via the hinge holes 86, 88, 92 and 94.

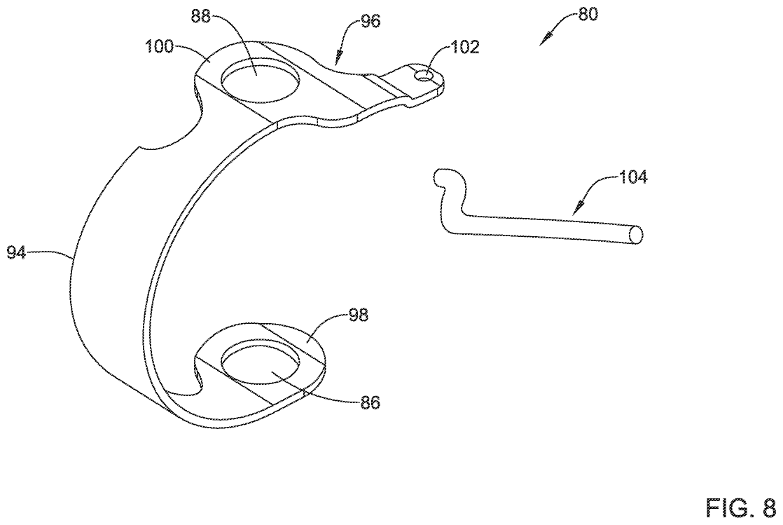

FIG. 8 is a perspective view of the first jaw 80. An actuator arm 96 extends from the first jaw 80 and in some cases may have a bent S shape in order to provide necessary clearance for other components such as but not limited to the first actuator member 44, the second jaw 82 and the body of the adaptor 38 itself. The first jaw 80 includes a first planar section 98 in which the first hinge hole 86 is formed and a second planar section 100 in which the second hinge hole 88 is formed. The actuator arm 96 extends from the second planar section 100 and includes an aperture 102 to accommodate the first actuator member 44. In some cases, the distal portion of the first actuator member 44 may include a distal Z-shaped tension member 104. The Z-shaped tension member 104 may simply be the distal end of the first actuator member 44, although in some cases the Z-shaped tension member 104 may be a separately formed part that is secured to the first actuator member 44.

It will be appreciated that the actuator arm 96 functions as a moment arm. If there is a desire to increase the cutting force that can be applied by the first jaw 80, one can simply make the actuator arm 96 longer. If less cutting force is desired, the actuator arm 96 may simply be made shorter, or the aperture 102 can be moved distally along the actuator arm 96, closer to the rest of the first jaw 80. In some cases, the first actuator member 44 (and the Z-shaped tension member 104) are metal wires that can be pushed or pulled to move the first jaw 80 in a desired direction.

With reference to FIGS. 6 and 7, it will be appreciated that the second jaw 82 similarly includes an actuator arm 108 that extends from the second jaw 82. The actuator arm 108 may have a bent S shape in order to provide necessary clearance for other components such as but not limited to the second actuator member 46, the first jaw 80 and the body of the adaptor 38 itself. The second jaw 82 includes a first planar section 110 in which the first hinge hole 92 is formed and a second planar section 112 in which the second hinge hole 94 is formed. The actuator arm 108 extends from the second planar section 112 and includes an aperture 114 to accommodate the second actuator member 46. In some cases, the distal portion of the second actuator member 46 may include a distal Z-shaped tension member similar to the Z-shaped tension member 104.

With reference back to FIG. 3, assembly of the biopsy device 32 is straightforward. The first actuator member 44 may be assembled to the aperture 102 of the first jaw 80 and a proximal end of the first actuator member 44 may be inserted into the first guide hole 72. The second actuator member 46 may be assembled to the aperture 114 of the second jaw 82 and a proximal end of the second actuator member 46 may be inserted into the second guide hole 74. The first jaw 80 and the second jaw 82 may be slightly squeezed together to allow placement of the jaws 80 and 82 over the hinge pins 66 and 68. Once aligned, the jaws 80 and 82 may be released to expand out or to snap over and onto the hinge pins 66 and 68.

In some instances, the first actuator member 44 and the second actuator member 46 may be operated independently in order to move each jaw 80 and 82 separately. In some instances, however, the first jaw 80 and the second jaw 82 are operated concurrently. The first actuator member 44 and the second actuator member 46 are crimped together (crimp 58, as shown in FIG. 2) in order to combine or join the actuator members 44 and 46 such that movement of one of the actuator members 44 and 46 applies the same movement to the other of the actuator members 44 and 46. The crimp 58 may exit the working channel 18 of the endoscope 10 as well as the handle body 50 and does not interfere with space in either case. In another example (not shown), the crimp 58 joins the first actuator member 44 and the second actuator member 46 at a distal end, and only one of the first actuator member 44 and the second actuator member 46 continues proximally through the endoscope 10 to be attached to the actuator handle 48.

Next, the crimped actuator member(s) 44 and 46 can be inserted into the working channel 18 of the endoscope 10 and out the proximal portion of the scope handle (not shown). The alignment marker 47 of the coupler 40 may be aligned to the working channel 18 of the endoscope 10 and can be secured in place relative to the distal tip region 16 of the endoscope 10. The crimped actuator member(s) 44, 46 can then be inserted into the handle connector and through the reinforced tube lumen and out the angled tube of the finger ring 54. The handle connector, distal to the tube, can then be slid over the actuator member(s) 44 and 46 to be connected to the proximal port 36 of the scope handle.

The finger ring 54 may be moved proximal to the alignment marker 62 and the crimped actuator member(s) 44 and 46 may be tensioned, displacing each the same distance in order to close the pair of jaws 42 evenly and centered. The fastener 60 may be tightened in order to engage the crimped actuator member(s) 44 and 46 within the finger ring 54, locking each of the actuator member(s) 44, 46 at the same length distance. In some cases, it is contemplated that the fastener 60 may be an electrical connector or plug that allows high frequency energy to be conducted through the actuator member(s) 44 and 46 for electrocautery cutting or hemostasis electrocauterization via the pair of jaws 42. In some cases, the actuator member(s) 44 and 46, may simply be bent around the tube to be secured.

In use, an operator may insert their thumb into the thumb ring 52 with the finger ring 54 gripped between their middle finger and their fore finger. Advancing the finger ring 54 forward (distally) opens the jaws 80 and 82 while retracting the finger ring 54 causes the jaws 80 and 82 to close together, thereby cutting, severing or otherwise capturing tissue.

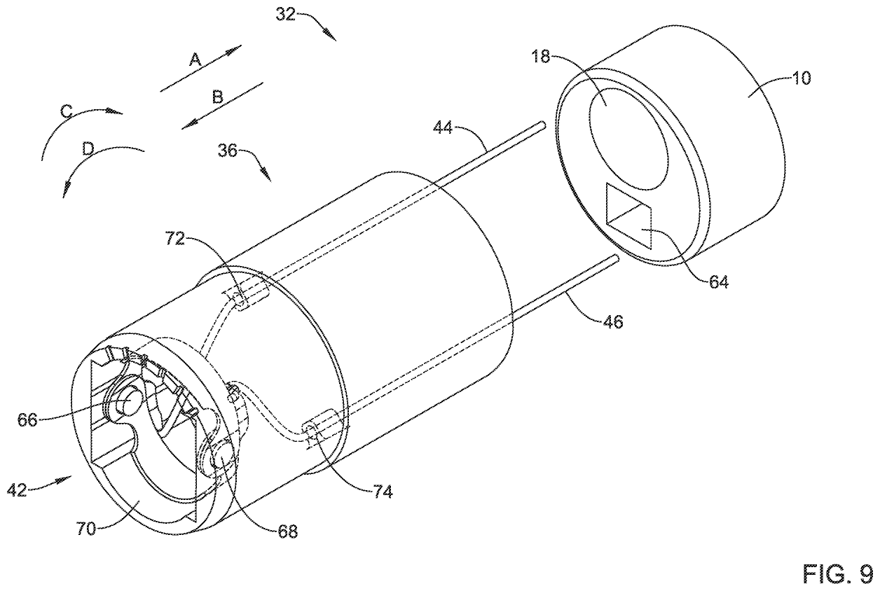

FIG. 9 is a perspective view of the biopsy device 32 shown with the jaws 80 and 82 shown in an opened configuration. The jaws 80 and 82 were moved into the open configuration by advancing the actuator members 44 and 46 in the direction of arrow B. This caused the first jaw 80 and its actuator arm 96 to rotate in the direction of arrow C while the second jaw 82 and its actuator arm 108 were caused to rotate in the direction of arrow D. If desired, suction may be applied through the working channel 18 of the endoscope 10 in order to draw tissue into the adaptor 38 to be cut, severed or captured. In some cases, a separate forceps may be directed through the working channel 18 in order to pull tissue into the adaptor 38. It will be appreciated that once the tissue has been cut, the closed jaws 80 and 82 can retain the tissue or sample within the adaptor 38 during removal from the body. The adaptor 38 may be able to store more than one sample, for example.

Once the biopsy or resection procedure has been completed, the fastener 60 can be loosened in order to release the actuator members 44 and 46 from the finger ring 54. The actuator handle 48 can be removed. The coupler 40 (and hence the adaptor 38) can be removed from the distal tip region 16 of the endoscope 10. The actuator member 44 and 46 can be withdrawn distally through the working channel 18. In some cases, rather than having both the first actuator member 44 and the second actuator member 46 both extend proximally to the actuator handle 48, one of the actuator members 44 and 46 may be crimped at a location proximal of the guide holes 72 and 74, and then only the other of the actuator members 44 and 46 can extend to the proximal end. This provides equal movement of both the jaws 80 and 82 without needing to equally set and align during assembly with the actuator handle 48. This can also save space within the working channel 18 for other uses. This can include providing additional space for other devices, and for flushing, for example.

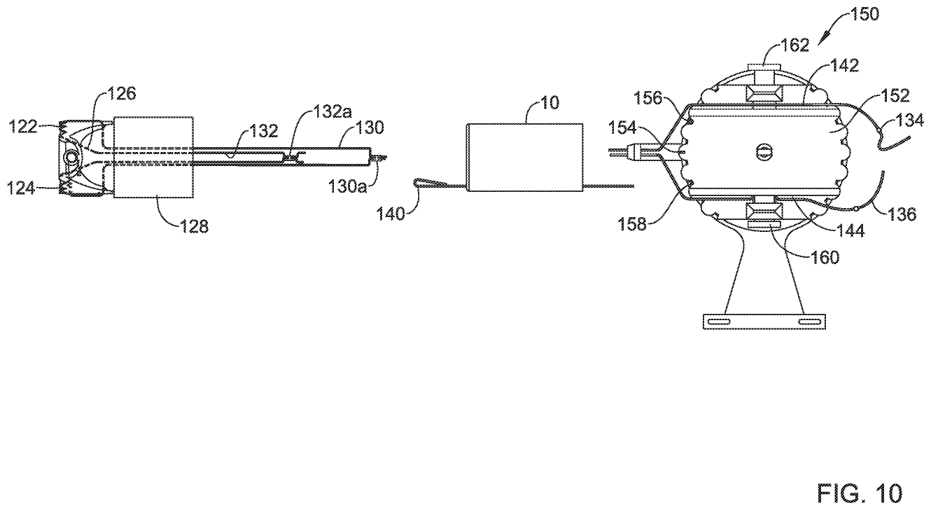

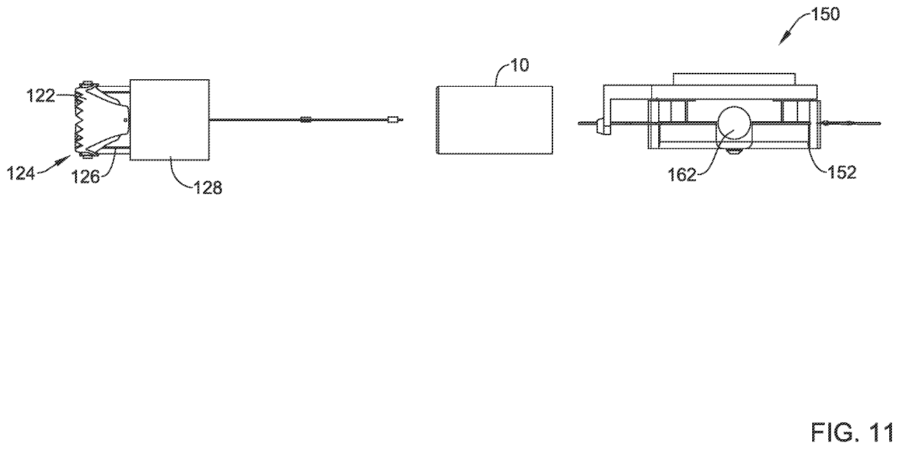

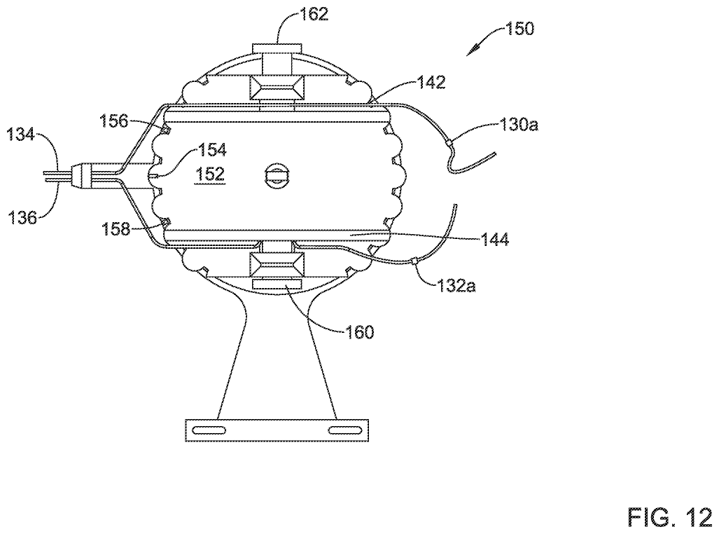

FIG. 10 is a side view of an illustrative biopsy device 120 while FIG. 11 is a top view of the illustrative biopsy device 120. FIG. 12 provides an enlarged view of an actuator handle 150, as will be discussed. The illustrative biopsy device 120 varies from the biopsy device 32 in that the jaws 122 and 124 are on the outside of an adaptor 126. A coupler 128 may be used to releasably secure the adaptor 126 to a reusable or disposable endoscope such as the endoscope 10. The biopsy device 120 utilizes tension members that are fiber cords or strings, lacking column strength. In FIG. 10, it can be seen that the tension members include an outer loop 130 and an inner loop 132. The outer loop 130 includes a first pair of tension members joined at a crimp 130a while the inner loop 132 includes a second pair of tension members joined at a crimp 132a. It will be appreciated that applying a tensile force to the outer loop 130 will cause the jaws 122 and 124 to open while applying a tensile force to the inner loop 132 will cause the jaws 122 and 124 to close.

Tension members 134 and 136 may be threaded through the working channel 18 of the endoscope 10 to an actuator handle 150 that includes a handle wheel 152. It will be appreciated that in some instances, the tension members 134 and 136 may represent single tension members. In some instances, the tension members 134 and 136 may instead each represent a loop, such as but not limited to the outer loop 130 and the inner loop 132. The tension members 134 and 136 may instead extend from the outer loop 130 and the inner loop 132. Rotating the handle wheel 152 counterclockwise opens the jaws 122 and 124. Rotating the handle wheel 152 clockwise, however, closes the jaws 122 and 124.

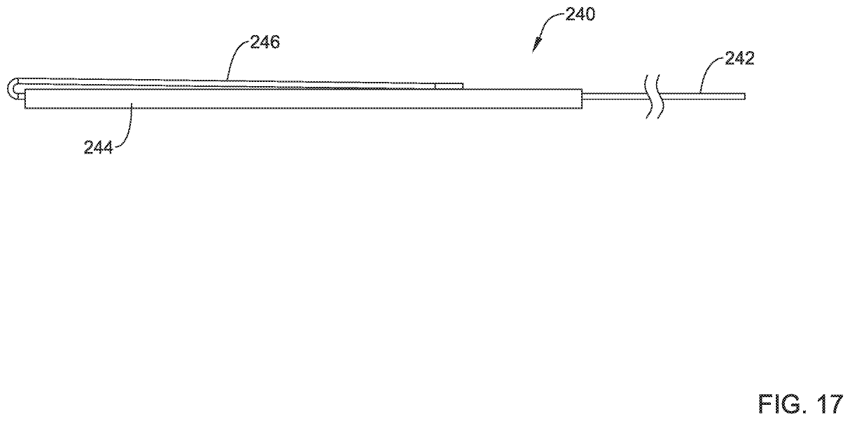

In some cases, as shown in FIG. 10, a wire hook 140 may be used to pull longer cords or loops through the working channel 18 of the endoscope 10. The wire hook 140 may be formed of stainless steel or the like, and can be removed when not needed. FIGS. 17-19 will provide further details regarding wire hooks such as the wire hook 140. In some cases, shorter color-coded hook wires may be used as extensions. To assemble the tension members to the actuator handle 130, the tension member 134 (associated with the outer loop 130) is laid into a slot 142. The tension member 136 (associated with the inner loop 132) is laid into a slot 144. The wheel 152 of the actuator handle 150 includes a wheel marking 154 while the actuator handle 150 includes a first marking 156 and a second marking 158. The wheel 152 is rotated in the direction of arrow D such that the wheel marking 154 aligns with the first marking 156 and the tension member 136 may be tensioned. A pin 160 is pushed in or threaded in to secure the tension member 136. The wheel 152 is then rotated in the direction of arrow C such that the wheel marking 154 aligns with the second marking 158 and the tension member 134 may be tensioned. A pin 162 is pushed in or threaded in to secure the tension member 134. When the wheel 152 is rotated such that the wheel marking 154 aligns with the first marking 156, the jaws 122 and 124 are closed. When the wheel 152 is rotated such that the wheel marking 154 aligns with the second marking 158, the jaws 122 and 124 are open.

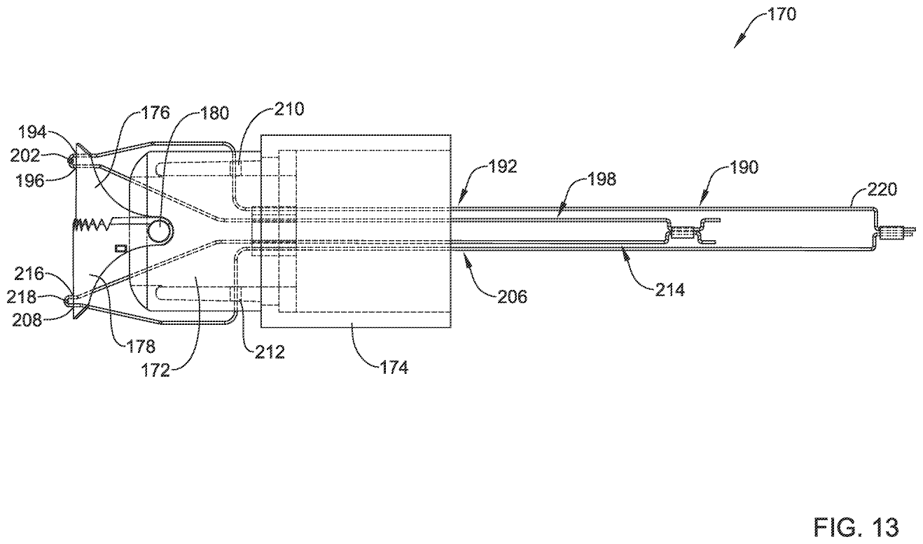

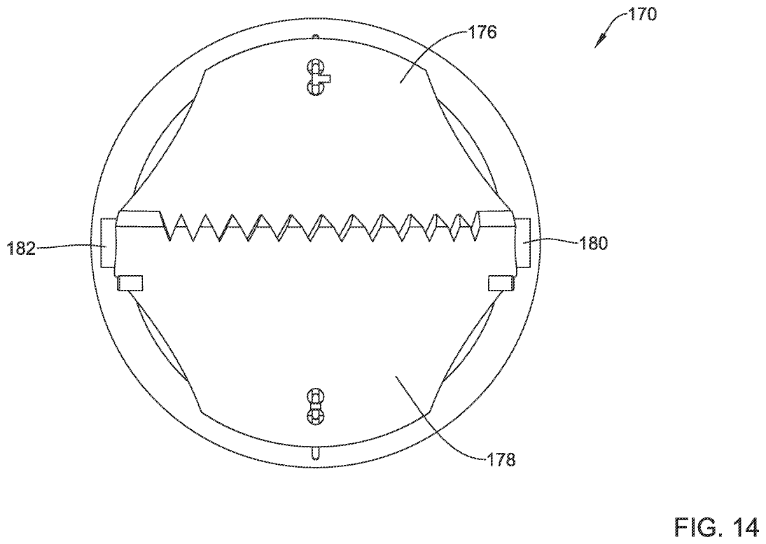

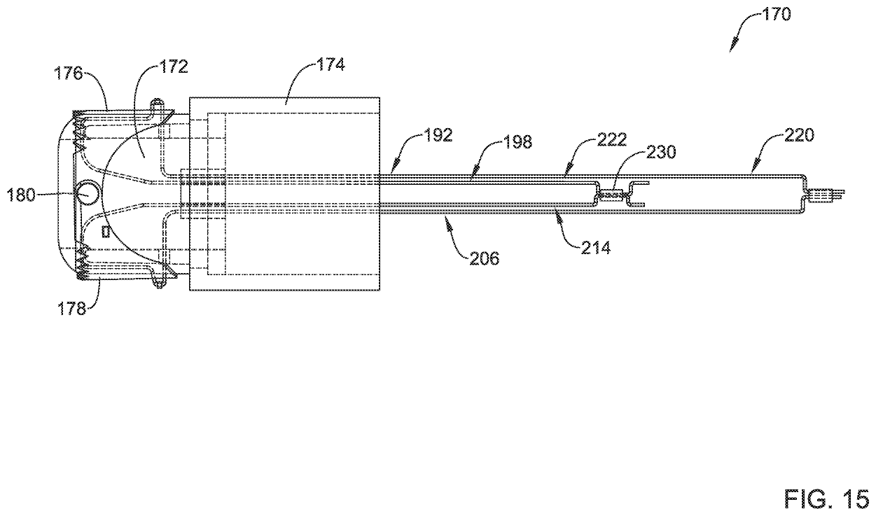

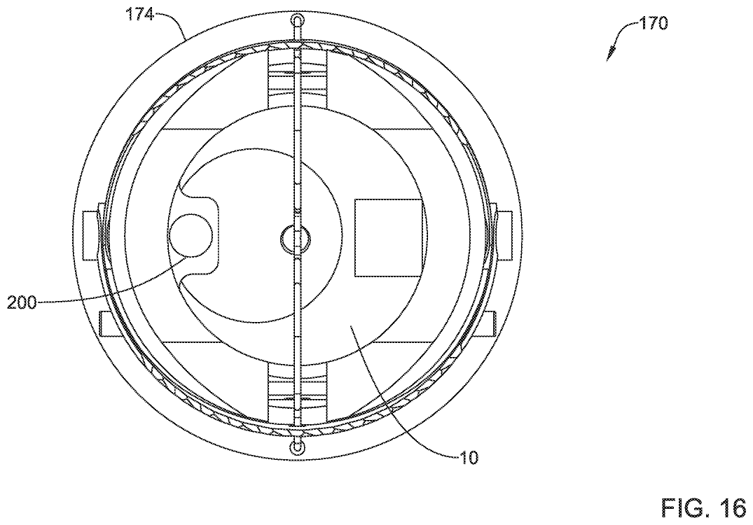

FIGS. 13 through 16 show an illustrative biopsy device 170. FIGS. 13 and 14 are side and end views, respectively, of the illustrative biopsy device 170 with its jaws closed. FIGS. 15 and 16 are side and end views, respectively, of the illustrative biopsy device 170 with its jaws open. The biopsy device 170 includes an adaptor 172 and a coupler 174 that is adapted to secure the adaptor 172 relative to the distal tip portion 16 of an endoscope 10. The biopsy device 170 includes a first jaw 176 and a second jaw 178. The adaptor 172 includes a first hinge pin 180 and a second hinge pin 182 that together pivotably secure the first jaw 176 and the second jaw 178 relative to the adaptor 172.

A fiber cord 190 can be assembled to the first jaw 176 and the adaptor 172 by first providing a crimp or knot at its mid-point. A leg 192 is threaded through the first jaw 176 at a hole 194 and then through an adaptor hole 210 and then extended proximally. A leg 198 can be threaded through a hole 196 and then through a guide hole 200 (FIG. 16), and then extended proximally. Instead of a crimp at crimp 202, the fiber cord 190 can be looped several times, or glued into position.

A fiber cord 204 can be assembled to the second jaw 178 and the adaptor 172 by first crimping a crimp or tying a knot at its mid-point. A leg 206 is threaded through the second jaw 178 through a hole 208 and then through an adaptor hole 212 and then extended proximally. A leg 214 can be extended through a hole 216 in the jaw 178 and then extended proximally. Instead of a crimp at 218, the fiber cord 206 can be looped several times, or glued into position. It will be appreciated that the crimp 202 cannot fit through the holes 194 and 196 of the first jaw 176. The crimp 218 cannot fit through the holes 208 and 216 of the second jaw 178. Accordingly, pulling an outer loop 220 pulls the legs 192 and 198 into the cup holes and pulls the crimp 202 against the first jaw 176 and pulls the crimp 218 against the second jaw 178 to open the jaws 176, 178.

With particular reference to FIG. 15, a crimp 230 is crimped to the legs 198 and 214 at a point where the legs 198 and 214 are the same length in order to form an inner loop 222. A crimp 232 is crimped to the legs 192 and 206 at a point where the legs 192 and 206 are the same length, in order to form the outer loop 220. The inner loop 222 can be the length of K while the outer loop 220 can be the length of M. In some cases, K and M can both be short as illustrated, or can be longer in order to extend to an actuator handle located at the proximal end. As illustrated, K is equal to 2 centimeters (cm) and M is equal to 4 cm. The biopsy device 170 may be used in conjunction with the actuator handle 150 described with respect to FIGS. 10 to 12, and can be connected to the actuator handle 150 in a similar manner.

With particular reference to FIG. 13, it will be appreciated that when the outer loop 220 is tensioned or displayed proximally, the legs 192 and 206 are displaced proximally to pull the first jaw 176 with the crimp 202 engaging the first jaw 176 and pulling towards the adaptor hole 210 and the second jaw 178 is pulled via the crimp 218 pulling towards the adaptor hole 212 to open the jaws 176 and 178. At the same time, a distal portion of the leg 198 is displaced between the first jaw 176 and an outer cup surface slot and a distal portion of the leg 214 is displaced between the second jaw 178 and the outer cup surface slot. The leg 198 and the leg 214 are wrapped around their respective slot edge when the jaws 176, 178 are fully open. When the inner loop 222 is tensioned or displaced proximally, the leg 198 and the leg 214 are displayed proximally and their respective leg portions between the jaws 176 and 178 and the adaptor 172 are pulled distally to move the first jaw 176 with engagement of the crimp 202 and the second jaw 178 is moved via engagement of the crimp 218 to close the jaws 176 and 178. At the same time, the leg 192 and the leg 206 are displayed distally as they are dragged by the crimp 202 and the crimp 218. It will be appreciated that the jaws 176 and 178 have a profile that is smaller than that of the coupler 174.

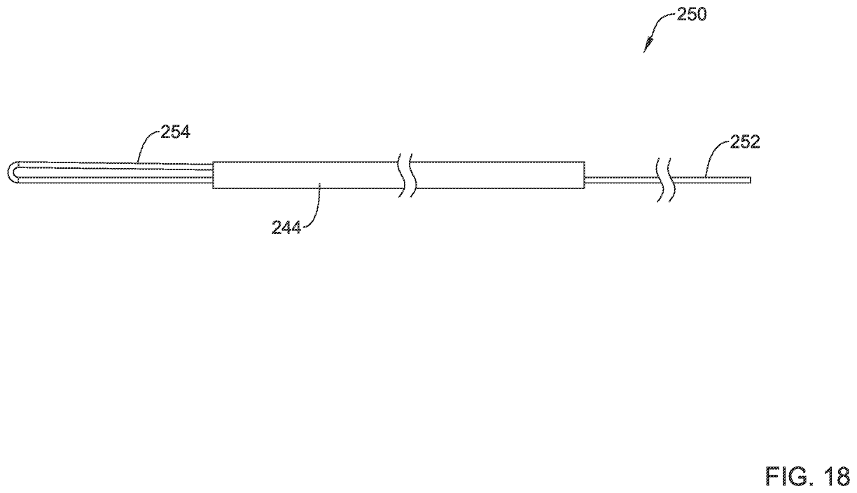

FIGS. 17 through 19 are side views of illustrative extension hook wires that may be used when threading actuator members, especially actuator and tension members that lack column strength, into and through the endoscope 10. In FIG. 17, an extension hook wire 240 includes a hook wire 242 and a color-coded locking tube 244 that is slidingly disposed over the hook wire 242. The hook wire 242 includes a terminal portion 246 that it outside of the color-coded locking tube 244. In some instances, the terminal portion 246 of the hook wire 242 bends back against an outer surface of the color-coded locking tube 244. In FIG. 18, an extension hook wire 250 includes a hook wire 252 having a terminal portion 254. In this case, the color-coded locking tube 244 covers a portion of the hook wire 252 as well as covering the terminal portion 254. While not illustrated, in some instances a proximal end of the color-coded locking tube 244 may be tapered in order to allow a smooth transition, e.g., to allow other devices to pass within the working channel 18 of the endoscope 10 without catching.

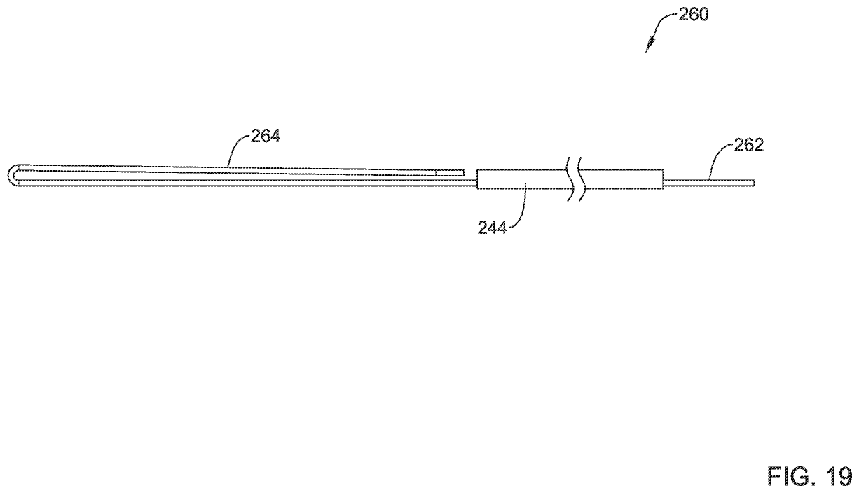

FIG. 19 illustrates use of the extension hook wires 240 and 250 with an assembly 260. An extension hook wire 262 includes a terminal portion 264. A tension member 266 includes a loop 268 that is formed via a knot or crimp 270. The loop 268 may be slid over the terminal portion 264 and the color-coded locking tube 244 may be slid down over the terminal portion 264 to secure the tension member 266 in place relative to the extension hook wire 262. The extension hook wire 240 and 250, and hence the tension member 266, may then be threaded into a desired position relative to the endoscope 10.

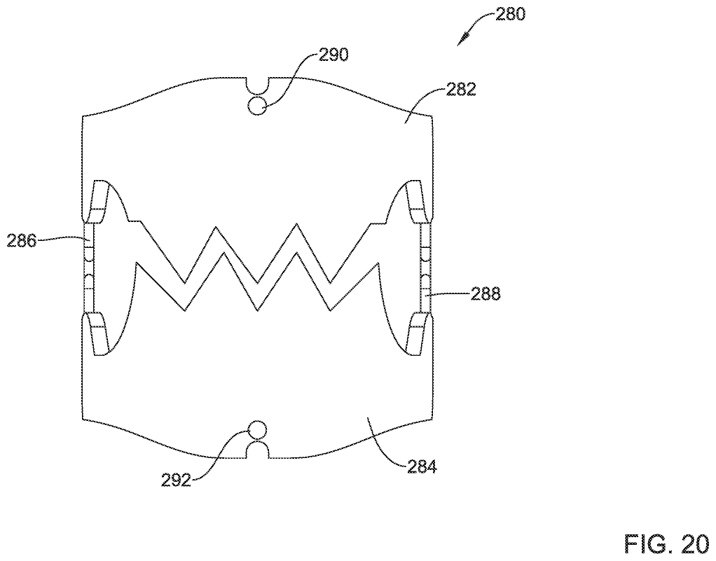

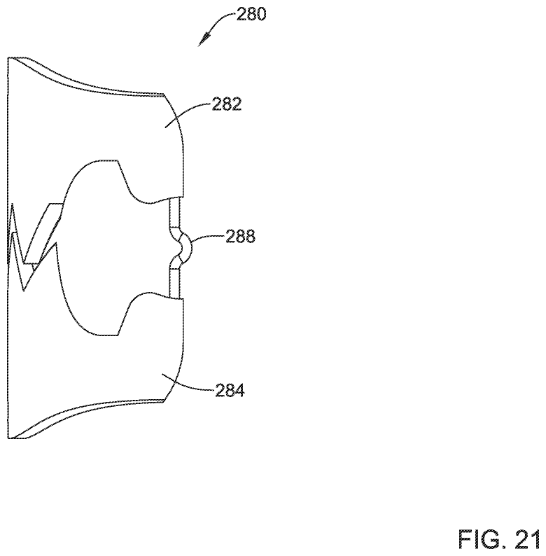

FIG. 20 is a front view and FIG. 21 is a side view of a pair of jaws 280. The pair of jaws 280 includes a first jaw 282 and a second jaw 284. A first spring 286 extends between the first jaw 282 and the second jaw 284. A second spring 288 extends between the first jaw 282 and the second jaw 284. The first jaw 282 includes a first hole 290 for securing a tension member and the second jaw 284 includes a second hole 292 for securing a tension member, as will be illustrated subsequently. The first and second springs 286 and 288 may be crimped, welded or glued onto the first jaw 282 and the second jaw 284. In some instances, the jaws 282 and 284 and the springs 286 and 288 may be laser cut from a single piece of U-shaped Nitinol. The springs 286 and 288 are adapted to bias the pair of jaws 280 into a closed position.

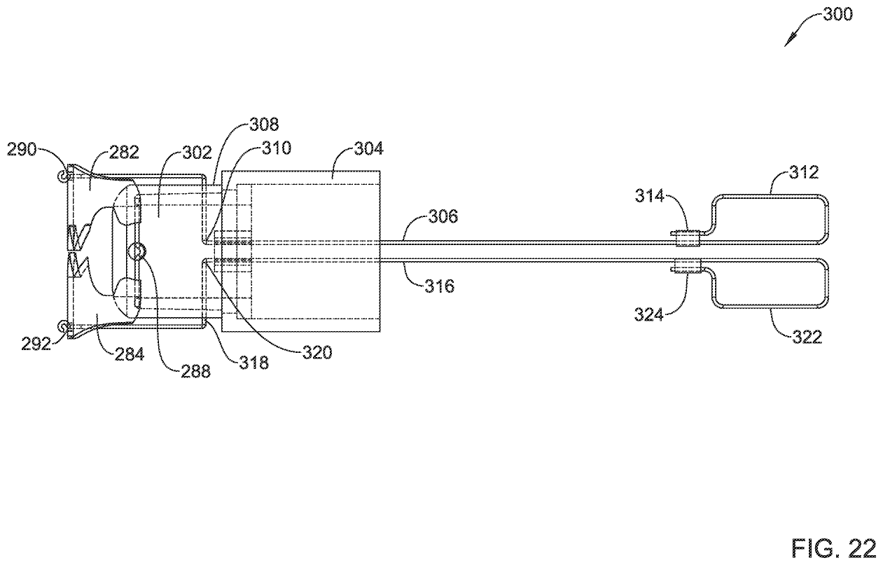

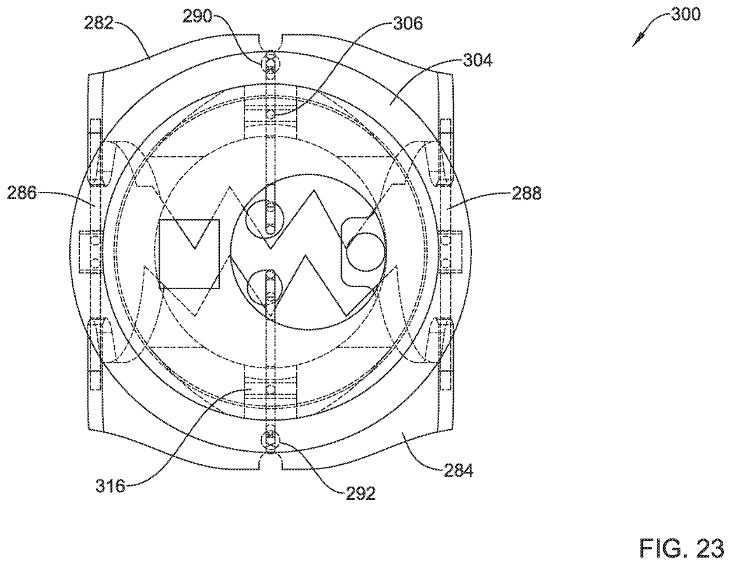

FIG. 22 is a side view of an illustrative biopsy device 300 and FIG. 23 is an end view thereof. The illustrative biopsy device 300 includes the first jaw 282 and the second jaw 284 as illustrated in FIGS. 20 and 21. The biopsy device 300 includes an adaptor 302 and a coupler 304 that is adapted to secure the adaptor 302 relative to the distal tip region 16 of the endoscope 10. A first tension member 306, which is a fiber cord, may be crimped, knotted or tied to the first jaw 282 via the first hole 290. The first tension member 306 is then assembled through a first cup hole 308 and through a guide hole 310 and extended proximally. In some instances, a loop 312 may be formed via a crimp 314. Similarly, a second tension member 316, which is also a fiber cord, may be crimped, knotted or tied to the second jaw 284 via the second hole 292. The second tension member 316 is then assembled through a second cup hole 318 and through a guide hole 320 and extended proximally. In some instances, a loop 322 may be formed via a crimp 324. The loops 312 and 322 may be secured to a single hook wire extension. In some cases, rather than forming the two loops 312 and 322, the crimp 314 may be used to simply crimp the first tension member 306 to the second tension member 316. In some cases, the crimp 314 may be used to connect a loop of a softer or more pliable material to the first tension member 306 and/or the crimp 324 may be used to connect a loop of a softer or more pliable material to the second tension member 316.

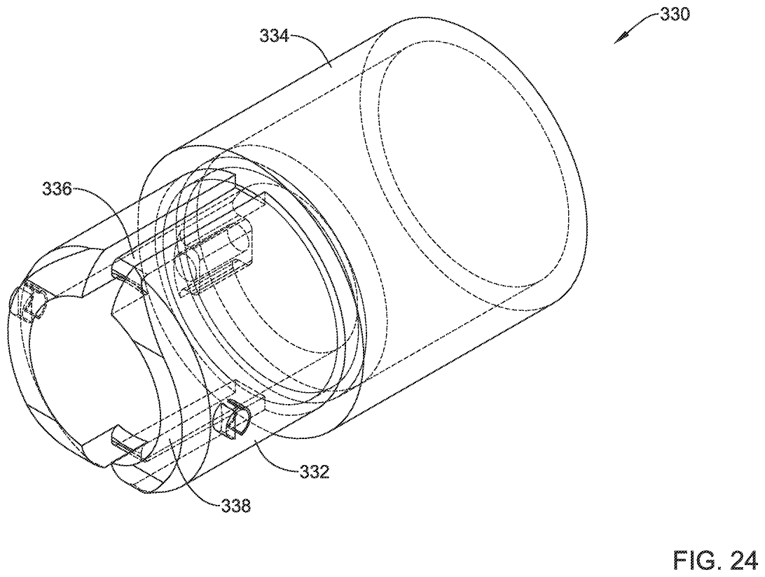

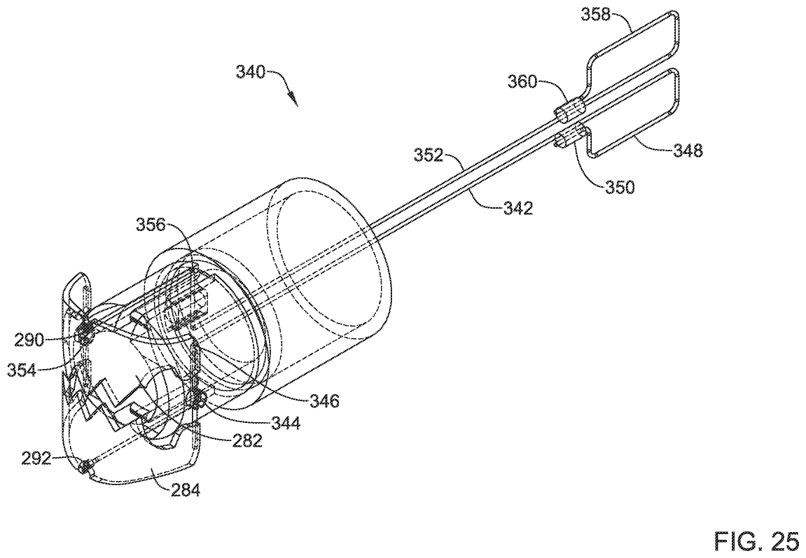

FIG. 24 is a perspective view of an assembly 330 that includes an adaptor 332 and a coupler 334 that is adapted to secure the adaptor 332 relative to the distal tip region 16 of the endoscope 10. The adaptor 332 includes recessed slots 336 and 338 that allow tissue samples to be suctioned into the adaptor 332. In some cases, if the recessed slots 336 and 338 were through slots, the tension members could then be metal wires that extend through the slots and are attached to the jaws. Since the metal wires can be used to both open and close the jaws 282 and 284, no springs would be needed. FIG. 25 shows an illustrative biopsy device 340 that utilizes metal wires.

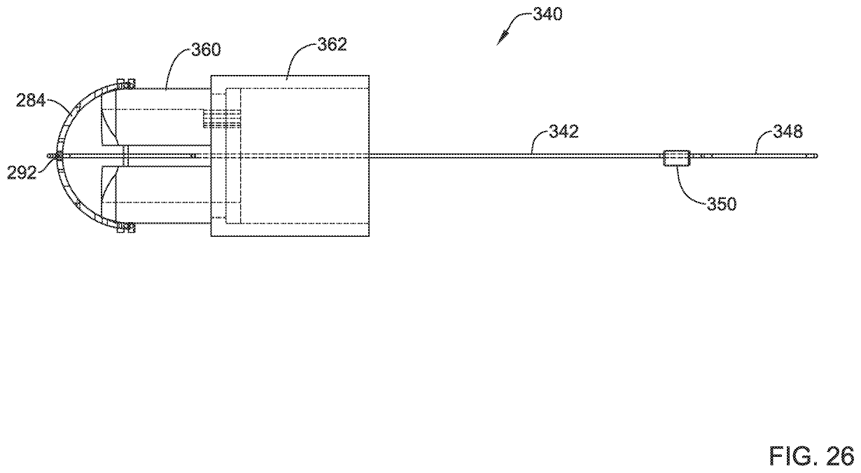

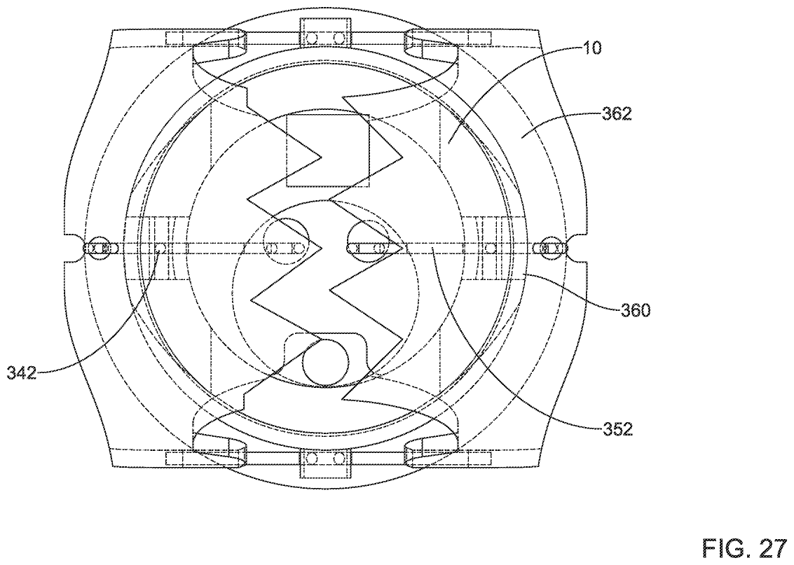

FIG. 25 is a perspective view of the illustrative biopsy device 340, FIG. 26 is a bottom view of the illustrative biopsy device 340 and FIG. 27 is an end view thereof. This is similar to the biopsy device 300 shown in FIG. 22, but without any springs. The biopsy device 340 includes an adaptor 360 and a coupler 362 that is adapted to secure the adaptor 360 relative to the distal tip region 16 of the endoscope 10. A first actuator member 342 is secured to the first jaw 282 via the first hole 290. The first tension actuator member 342 is then assembled through a first cup hole 344 and through a guide hole 346 and extended proximally. In some instances, a loop 348 may be formed via a crimp 350. Similarly, a second actuator member 352 is secured to the second jaw 284 via the second hole 292. The second actuator member 352 is then assembled through a second cup hole 354 and through a guide hole 356 and extended proximally. In some instances, a loop 358 may be formed via a crimp 360.

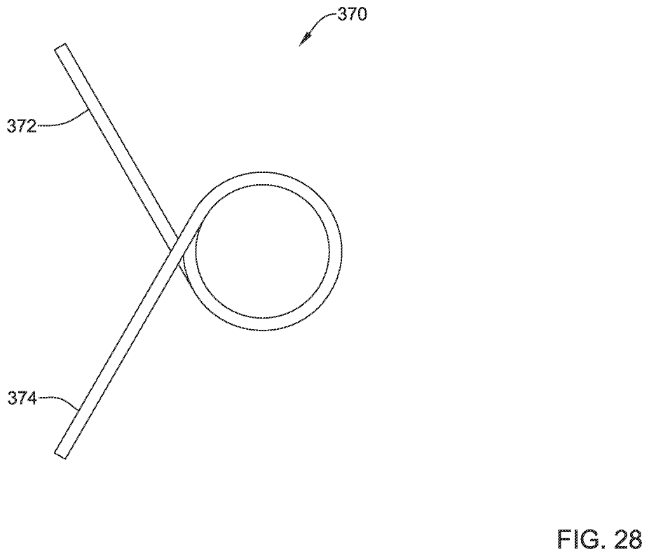

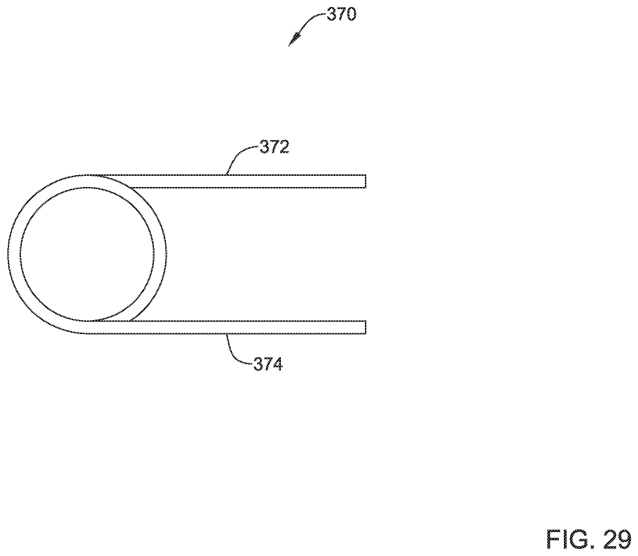

FIG. 28 is a schematic view of a torsion spring 370 that may be used instead of the springs 286 and 288 in the biopsy device 300 best seen in FIG. 22. The torsion spring 370 has its legs 372 and 374 at or beyond vertical at equilibrium, and may be used in the biopsy device 300 to keep the jaws 282 and 284 in a normally closed position. In some instances, FIG. 29 may be considered as showing the shape of the torsion spring 370 when deflected to open the jaws 282 and 284. Alternatively, FIG. 29 may be considered as showing a desired shape of the torsion spring 370 at equilibrium that may be used in the biopsy device 300 in order to hold the jaws 282 and 284 at a normally open position. In this case, FIG. 28 would represent the torsion spring 370 deflected into a position in which the jaws 282 and 284 are closed.

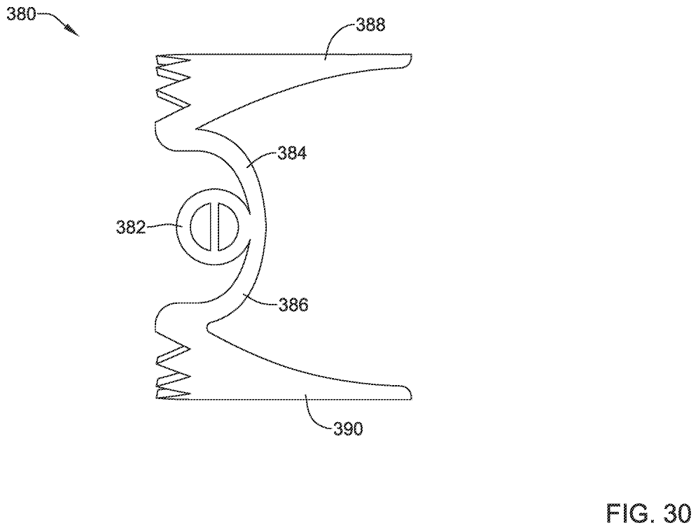

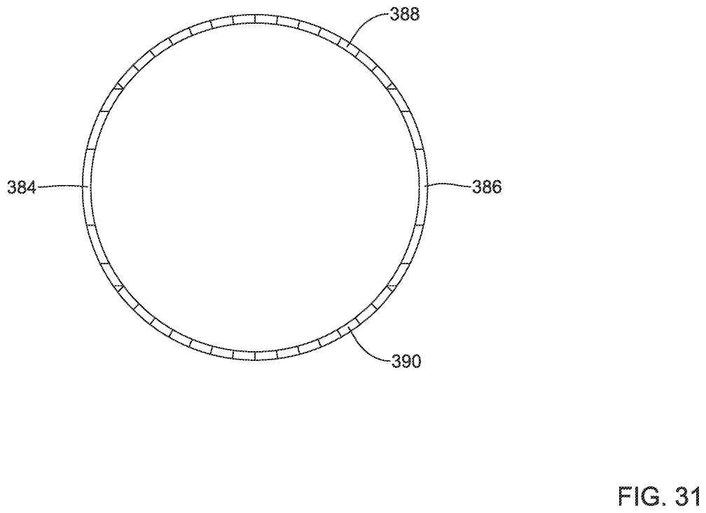

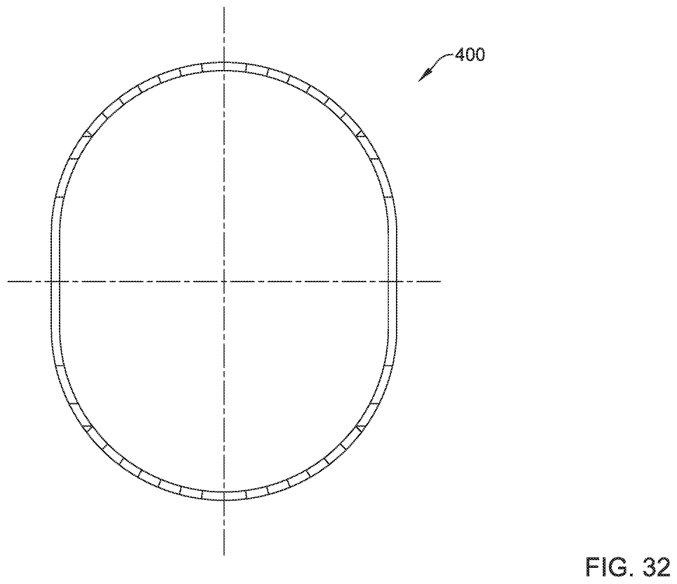

FIG. 30 is a side view of a pair of jaws 380 that are normally open. The pair of jaws 380 includes a center anchor 382 and a pair of spring arms 384 and 386 extending from the center anchor 382. A first jaw 388 extends from the spring arm 384 and a second jaw 390 extends from the spring arm 386. The center anchor 382 remains stationary while the spring arms 384 and 386 deflect to allow the jaws 388 and 390 to move. The pair of jaws 380 may be formed by laser cutting a Nitinol tube. FIG. 31 shows laser cutting a circular Nitinol tube to form the pair of jaws 380. FIG. 32 shows laser cutting a racetrack-shaped Nitinol tube 400 to form the pair of jaws 380.

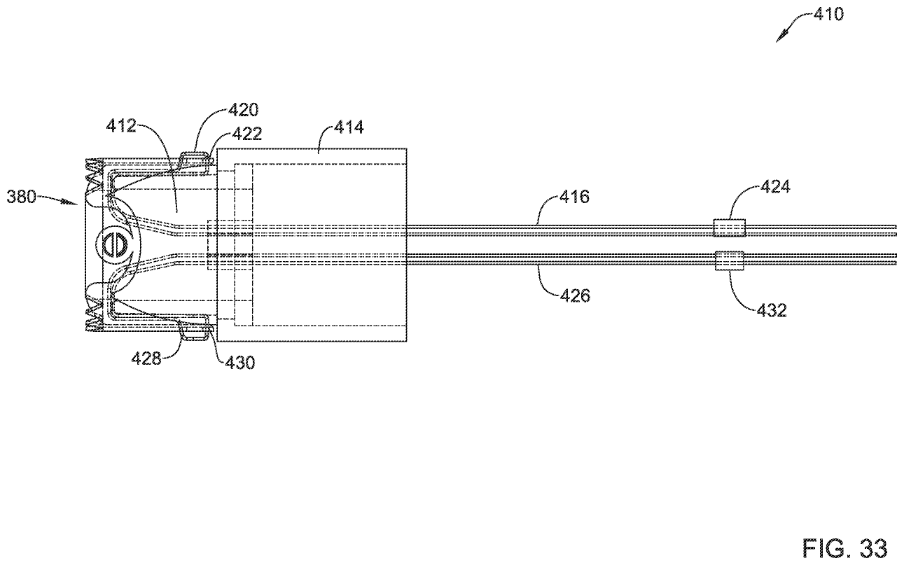

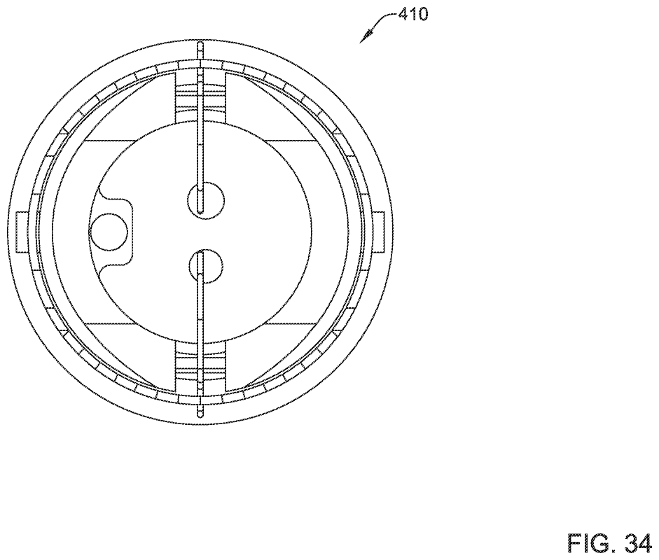

FIGS. 33 and 34 are side and end views, respectively, of an illustrative biopsy device 410 that includes the illustrative pair of jaws 380 shown in FIG. 30. The pair of jaws 380 is secured relative to an adaptor 412. A coupler 414 is adapted to secure the adaptor 412 relative to the distal tip region 16 of the endoscope 10. A tension member 416 may be assembled to the first jaw 388 by inserting one end of the tension member 416 into a hole 420 and inserting the other end of the tension member 416 into a hole 422. Both ends are then extended proximally relative to the first jaw 388 and may be crimped together via a crimp 424. A tension member 426 may be assembled to the second jaw 390 by inserting one end of the tension member 426 into a hole 428 and inserting the other end of the tension member 426 into a hole 430. Both ends are then extended proximally relative to the second jaw 390 and may be crimped together via a crimp 432.

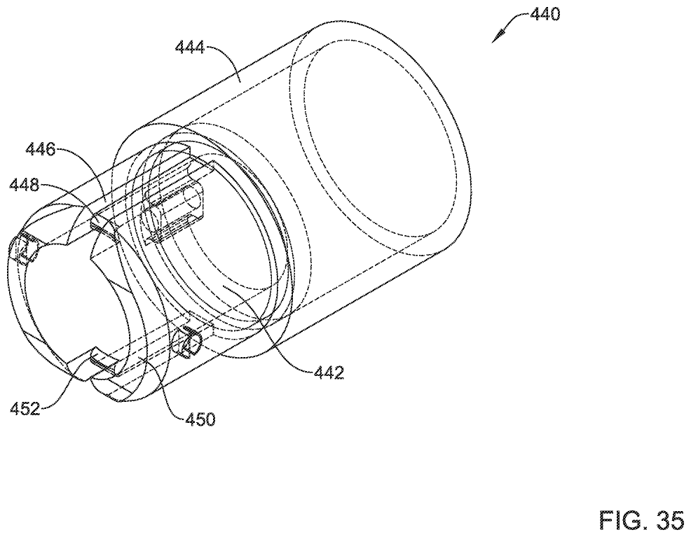

FIG. 35 is a perspective view of an illustrative assembly 440 that includes an adaptor 442 and a coupler 444 that is adapted to secure the adaptor 442 to the distal tip region 16 of the endoscope 10. The adaptor 442 includes a first recessed slot 446 having a first slot edge 448 and a second recessed slot 450 having a second slot edge 452. It will be appreciated that once the tension member 406 has been fed through the holes 420 and 422, the tension member 406 may wrap around the first slot edge 448 and pass through the first recessed slot 446 as it extends proximally. Similarly, once the two ends of the tension member 426 have been fed through the holes 428 and 430, the tension member 426 may wrap around the second slot edge 452 and pass through the second recessed slot 460 as it extends proximally.

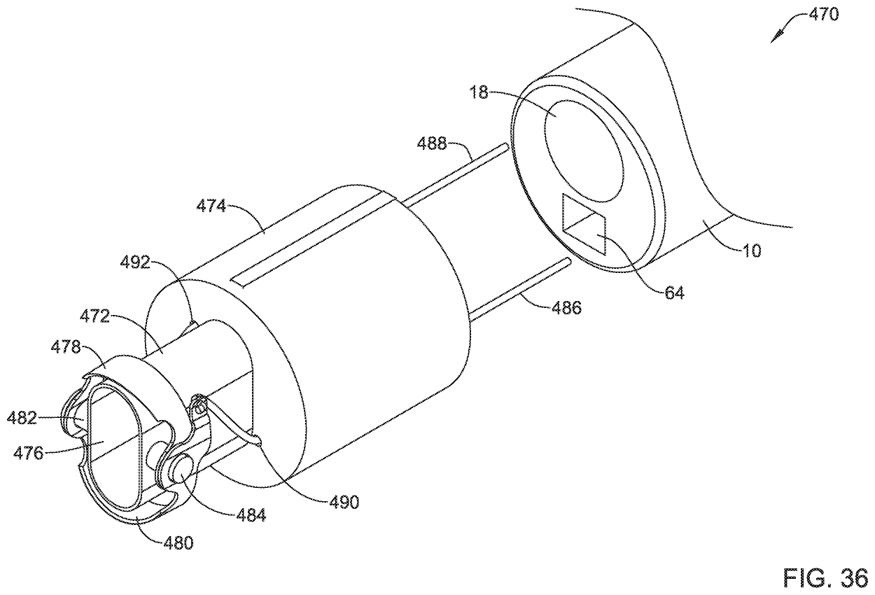

FIG. 36 is a perspective view of an illustrative biopsy device 470. The illustrative biopsy device 470 includes an adaptor 472 and a coupler 474 that is adapted to secure the adaptor 472 to the distal tip region 16 of the endoscope 10. The adaptor 472 provides a suction channel 476 that extends through an interior of the adaptor 472. The suction channel 476 enables a source of vacuum to be used to pull tissue towards and into the suction channel 476 such that a first jaw 478 and a second jaw 480, which are pivotably secured to a pair of hinge pins 482 and 484, are able to move from an open position (as shown) to a closed position in which the jaws 478 and 480 cut off a tissue sample. In some cases, the jaws 478 and 480 have a construction similar to the pair of jaws 42 shown for example in FIGS. 6 and 7.

A first actuation member 486 is operably coupled to the jaw 480 and a second actuation member 488 is operably coupled to the jaw 478. The first actuation member 486 and the second actuation member 488 extend proximally from the jaws 478 and 480 and pass through a pair of holes 490 and 492, respectively, that are formed within a panel 494 that seals off a distal end of the coupler 474. The holes 490 and 492 are sealed with an elastomeric material around the first actuation member 486 and the second actuation member 488 to provide a fluid-tight seal around the actuation members 486 and 488. In some cases, the panel 494 may be formed of an elastomeric material that stretches to allow the actuation members 486 and 488 to pass therethrough. The actuation members 486 and 488 extend proximally through the working channel 18 of the endoscope 10.

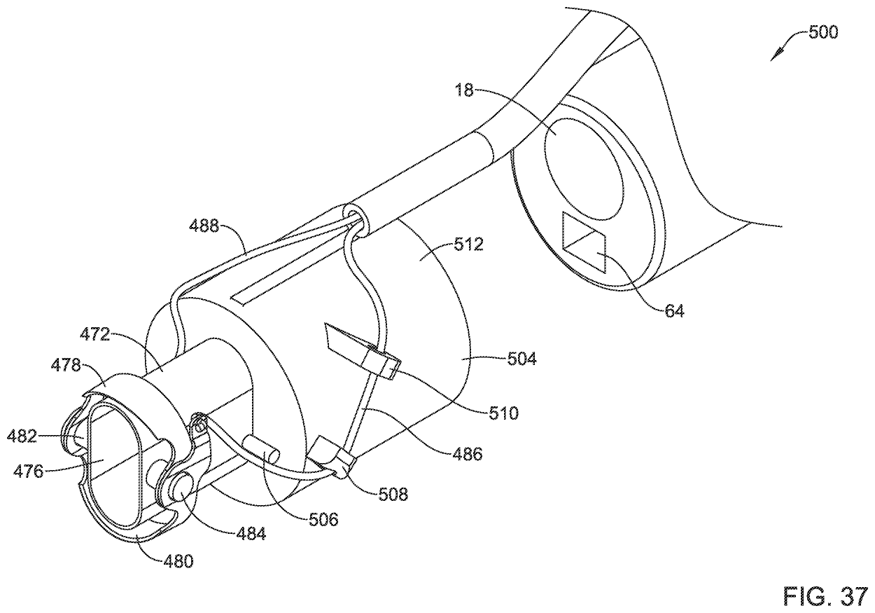

FIG. 37 is a perspective view of an illustrative biopsy device 500. The illustrative biopsy device 500 includes an adaptor 502 and a coupler 504 that is adapted to secure the adaptor 502 to the distal tip region 16 of the endoscope 10. The biopsy device 500 is similar to the biopsy device 470 shown in FIG. 36, but in FIG. 37 the adaptor 502 and the coupler 504 include additional structures that guide the actuation members 486 and 488 proximally from the jaws 478 and 480. The adaptor 502 includes a guide pin 506 that helps to direct the actuation member 486. While not visible, another guide pin on the opposite side of the adaptor 502 helps to direct the actuation member 486.

The coupler 504 includes a first guide hole 508 and a second guide hole 510 that are formed within tabs projecting from the coupler 504. The guide holes 508 and 510 may be positioned on an exterior surface 512 of the coupler 504. The guide pin 506 and the guide holes 508 and 510 in combination help to guide the actuation member 486 from the jaw 480 and into an external tube 514 that extends proximally and externally to the endoscope 10. While not visible, it will be appreciated that the opposing side of the coupler 504 includes similar guide holes to guide and direct the actuation member 488 from the jaw 478 and into the external tube 514.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A biopsy device adapted for use with an endoscope, the biopsy device comprising:
an adaptor adapted to be secured relative to an endoscope, the adaptor including an inner surface and an outer surface;
a pair of jaws pivotably disposed relative to the adaptor; and
an actuator operably coupled with the pair of jaws and extending proximally therefrom, the actuator adapted to move the pair of jaws relative to the adaptor.

2. The biopsy device of claim 1, wherein the actuator is adapted to be moved in a first axial direction to open the pair of jaws and to be moved in an opposing second axial direction to close the jaws.

3. The biopsy device of claim 1, wherein the pair of jaws are biased to a first position, and the actuator is adapted to move the pair of jaws against a biasing force.

4. The biopsy device of claim 1, wherein the adaptor comprises a first pivot point along a first side of the biopsy device housing and a second pivot point along a second side of the biopsy device housing.

5. The biopsy device of claim 4, wherein the first pivot point and the second pivot point are disposed on the inner surface of the adaptor.

6. The biopsy device of claim 4, wherein the first pivot point and the second pivot are disposed on the outer surface of the adaptor.

7. The biopsy device of claim 1, wherein the actuator comprises a first actuator member secured relative to the first jaw and a second actuator member secured relative to the second jaw such that applying an axial force to the first actuator member and/or the second actuator member causes the first jaw and/or the second jaw to pivot relative to the biopsy device housing.

8. The biopsy device of claim 7, wherein the first jaw includes a first actuator arm and the second jaw includes a second actuator arm, with the first actuator member secured to the first actuator arm and the second actuation member secured to the second actuator arm.

9. The biopsy device of claim 7, wherein the adaptor includes a first alignment feature that accommodates the first actuator member extending therethrough and a second alignment feature that accommodates the second actuator member extending therethrough.

10. The biopsy device of claim 1, further comprising a coupler adapted to releasably secure the biopsy device housing relative to the endoscope.

11. The biopsy device of claim 1, further comprising an actuator handle operably coupled with the actuator member.

12. The biopsy device of claim 11, wherein the actuator handle includes a first member slidingly secured relative to a second member, with the actuator securable to the first member, wherein moving the first member relative to the second member causes the actuator to open or close the pair of jaws.

13. The biopsy device of claim 11, wherein the actuator handle includes a rotatable wheel, with the actuator securable to the rotatable wheel, and rotating the wheel causes the actuator to open or close the pair of jaws.

14. A biopsy device adapted for use with an endoscope, the biopsy device comprising:
an adaptor adapted to be secured relative to an endoscope;
a first jaw pivotably secured to the adaptor;
a second jaw pivotably secured to the adaptor, the first jaw and the second jaw together pivotable to a closed configuration in which the first jaw and the second jaw cooperate to cut tissue;
a first actuator member secured to the first jaw such that moving the first actuator member causes the first jaw to pivot relative to the adaptor;
a second actuator member secured to the second jaw such that moving the second actuator member causes the second jaw to pivot relative to the adaptor; and
an actuator handle operably coupled with the first actuator member and the second actuator member such that manipulating the actuator handle causes the first jaw and the second jaw to pivot relative to the adaptor.

15. The biopsy device of claim 14, wherein the first actuator member and the second actuator member extend proximally to the actuator handle.

16. The biopsy device of claim 14, wherein the first actuator member and the second actuator member are coupled together, and only one of the first actuator member and the second member extend proximally to the actuator handle.

17. The biopsy device of claim 14, wherein the actuator handle includes a first handle slidingly secured relative to a second handle, with at least one of the first actuator member and the second actuator handle securable to the first member, wherein moving the first handle relative to the second handle causes the actuator to open or close the pair of jaws.

18. The biopsy device of claim 14, wherein the actuator handle includes a rotatable wheel, with the first actuator member and the second actuator member securable to the rotatable wheel, and rotating the wheel causes the actuator to open or close the pair of jaws.

19. A medical device adapted for use with an access device, the medical device comprising:
an adaptor adapted to be secured relative to an access device;
a pair of jaws pivotably secured to the adaptor, the pair of jaws movable between an open position in which each of the pair of jaws pivots away from each other and a cutting position in which each of the pair of jaws pivots toward each other; and
a spring biasing the pair of jaws towards one of the open position and the cutting position; and
an actuator operably coupled with the pair of jaws and extending proximally therefrom, the actuator adapted to move the pair of jaws against a biasing force applied by the spring.

20. The medical device of claim 19, further comprising an actuator handle operably coupled with the actuator.
